(19) United States
(12) Patent Application Publication
(10) Pub. No.: US 2025/0200360 A1
(43) Pub. Date: Jun. 19, 2025

(54) METHOD AND APPARATUS WITH ORGANIC MOLECULE SPECTRUM PREDICTION

(71) Applicants: Samsung Electronics Co., Ltd., Suwon-si (KR); La Corporation de l'École des Hautes Études Commerciales de Montréal, Montréal (CA)

(72) Inventors: Hasup LEE, Suwon-si (KR); Sang Ha PARK, Suwon-si (KR); Kuhwan JEONG, Suwon-si (KR); Jian TANG, Montreal (CA); Ki Soo KWON, Suwon-si (KR); Miyoung JANG, Suwon-si (KR); Eun Hyun CHO, Suwon-si (KR)

(73) Assignees: Samsung Electronics Co., Ltd., Suwon-si (KR); La Corporation de l'École des Hautes Études Commerciales de Montréal, Montréal (CA)

(21) Appl. No.: 18/644,296

(22) Filed: Apr. 24, 2024

(30) Foreign Application Priority Data
Dec. 13, 2023 (KR) 10-2023-0181066

(51) Int. Cl. G06N 3/08; G06N 5/04

(52) U.S. Cl. CPC G06N 3/08; G06N 5/04

(57) ABSTRACT

A method and apparatus with organic molecule spectrum prediction are disclosed. The method includes accessing a molecular structure representation of an organic molecule; generating parameters of an approximated Franck-Condon progression by inputting the molecular structure representation to a neural network model that infers the parameters from the molecular structure representation; and generating a spectrum of the organic molecule based on the generated parameters.

### CROSS-REFERENCE TO RELATED APPLICATIONS

This application claims the benefit under 35 USC § 119(a) of Korean Patent Application No. 10-2023-0181066, filed on Dec. 13, 2023, in the Korean Intellectual Property Office, the entire disclosure of which is incorporated herein by reference for all purposes.

### BACKGROUND

### 1. Field

The following embodiment relates to a method and apparatus with organic molecule spectrum prediction.

### 2. Description of Related Art

When an organic molecule receives electrical and light energy, the organic molecule may transition from a ground state to an excited state and may emit light when the organic molecule transitions from the excited state to the ground state. Due to a difference in molecular structure between the ground state and the excited state, light may be absorbed or emitted in a wide wavelength range rather than at a particular wavelength, as is usually the case for a molecule not subjected to excitation.

Due to the wide absorption and emission wavelength range, various pieces of data such as maximum absorption and emission wavelengths (λ_{abs,max}, λ_{emi,max}), a half-width (σ_{abs}, σₑₘᵢ), an area, and a mean emission energy (Em50/50) may be calculated and used as data and information (or features) for training a machine learning model to predict absorption and emission spectra.

Conventional technical models related to determining absorption and emission spectra may not completely or accurately predict a wide spectrum of an organic molecule, and may predict abbreviated information, such as maximum absorption and emission wavelengths, a half-width, and an area, or may make predictions by discretizing spectrum information. Such abbreviated information may not completely express the spectrum of an organic molecule that absorbs and emits in a wide range.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a method is performed by a computing device that includes processing hardware and storage hardware, and the method includes: storing, in the storage hardware, a representation of a molecular structure of an organic molecule; performing, by a neural network model executed by the processing hardware, inference on the representation of the molecular structure, to infer parameters of an approximated Franck-Condon progression; and generating a spectrum of the organic molecule by the processing hardware applying the inferred parameters to the approximated Franck-Condon progression.

The method may further include training the neural network model based on an error between a ground truth spectrum of the representation of the molecular structure and the generated spectrum of the organic molecule.

With respect to organic molecules, the neural network model may be trained after calculating a highest occupied molecular orbital (homo) energy level, a lowest unoccupied molecular orbital (lumo) energy level, and an electron transition energy level through a density functional theory (DFT).

The processing hardware may be configured to perform the following as the approximated Franck-Condon progression:

$$I ( x ) = {\sum }_{{v}_{1} = 0}^{∞} {\sum }_{{v}_{2} = 0}^{∞} … {\sum }_{{v}_{n} = 0}^{∞} {( \frac{{E}_{0} - {\sum }_{i = 1}^{n} {v}_{i} {ω}_{i}}{{E}_{0}} )}^{3} {\prod }_{i = 1}^{n} ( \frac{{S}_{i}^{{v}_{i}} {e}^{- {S}_{i}}}{{v}_{i} !} ) exp [ - \frac{{\{ x - ( {E}_{0} - {\sum }_{i = 1}^{n} {v}_{i} {ω}_{i} ) \}}^{2}}{2 {C}^{2}} ] ,$$

where, I(x) denotes an intensity of absorption and emission of a wavelength x, E₀ denotes electron transition energy in a ground state and an excited state, Sᵢ denotes a Huang-Rhys factor that is a difference in molecular structure expressed by a specific vibrational mode, ωᵢ denotes a vibrational energy level of a specific vibrational mode, vᵢ denotes an energy level of a specific vibrational mode, C denotes a baseline distribution function parameter, and n denotes an approximated number of vibrational energy levels.

The parameters may include transition energy of the Franck-Condon progression, a baseline distribution function parameter, transition degrees, and vibrational energy levels of each vibrational mode according to the approximated number of vibrational energy levels.

The molecular structure may be in a Molfile format or a format of the simplified molecular-input line-entry system (SMILES).

The spectrum of the organic molecule may include an emission or absorption spectrum according to a wavelength with respect to the organic molecule.

In another general aspect, a method of training a neural network model is performed by a computing device that includes processing hardware and storage hardware, and the method includes: accessing, by the processing hardware, training data in the storage hardware, the training data including training samples, each training sample including a molecular structure representation of an organic molecule and an associated ground truth spectrum of the molecular structure; for each training sample, generating, by the processing hardware, corresponding parameters of an approximated Franck-Condon progression by inputting the molecular structure representation of the training sample to a neural network model which infers the corresponding parameters; for each training sample, generating, by the processing hardware, a corresponding spectrum of the training sample based on the corresponding obtained parameters; and for each training sample, training, by the processing hardware, the neural network model based on an error between the corresponding ground truth spectrum and the corresponding generated spectrum.

The processing hardware may be configured to perform the following as the approximated Franck-Condon progression:

$$I ( x ) = {\sum }_{{v}_{1} = 0}^{∞} {\sum }_{{v}_{2} = 0}^{∞} … {\sum }_{{v}_{n} = 0}^{∞} {( \frac{{E}_{0} - {\sum }_{i = 1}^{n} {v}_{i} {ω}_{i}}{{E}_{0}} )}^{3} {\prod }_{i = 1}^{n} ( \frac{{S}_{i}^{{v}_{i}} {e}^{- {S}_{i}}}{{v}_{i} !} ) exp [ - \frac{{\{ x - ( {E}_{0} - {\sum }_{i = 1}^{n} {v}_{i} {ω}_{i} ) \}}^{2}}{2 {C}^{2}} ] ,$$

where, I(x) denotes an intensity of absorption and emission of a wavelength x, E₀ denotes electron transition energy in a ground state and an excited state, Sᵢ denotes a Huang-Rhys factor that is a difference in molecular structure expressed by a specific vibrational mode, ωᵢ denotes a vibrational energy level of a specific vibrational mode, vᵢ denotes an energy level of a specific vibrational mode, C denotes a baseline distribution function parameter, and n denotes an approximated number of vibrational energy levels.

The parameters may include transition energy of the Franck-Condon progression, a baseline distribution function parameter, transition degrees, and vibrational energy levels of each vibrational mode according to an approximated number of vibrational energy levels.

The obtaining of the parameters for each of the training samples may include: calculating a highest occupied molecular orbital (homo) energy level, a lowest unoccupied molecular orbital (lumo) energy level, and an electron transition energy level using a density functional theory (DFT) for the organic molecules.

The molecular structure representation of each training sample may be in a Molfile format or a format of the simplified molecular-input line-entry system (SMILES).

The neural network model may include layers of nodes with weights of connections therebetween, and the layers may include an input layer configured to receive the molecule structure representations and an output layer configured to output the parameters.

In another general aspect, an apparatus includes: one or more processors; and a memory storing instructions configured to cause the one or more processors to: access a molecular structure representation of an organic molecule; generate parameters of an approximated Franck-Condon progression by inputting the molecular structure representation to a neural network model that infers the parameters from the molecular structure representation; and generate a spectrum of the organic molecule based on the generated parameters.

The neural network model may be trained based on an error between a ground truth spectrum of the molecular structure representation and the generated spectrum.

With respect to organic molecules, the neural network model may be trained after calculating a highest occupied molecular orbital (homo) energy level, a lowest unoccupied molecular orbital (lumo) energy level, and an electron transition energy level through a density functional theory (DFT).

The instructions may be further configured to cause the one or more processors to perform the following approximated Franck-Condon progression:

$$I ( x ) = {\sum }_{{v}_{1} = 0}^{∞} {\sum }_{{v}_{2} = 0}^{∞} … {\sum }_{{v}_{n} = 0}^{∞} {( \frac{{E}_{0} - {\sum }_{i = 1}^{n} {v}_{i} {ω}_{i}}{{E}_{0}} )}^{3} {\prod }_{i = 1}^{n} ( \frac{{S}_{i}^{{v}_{i}} {e}^{- {S}_{i}}}{{v}_{i} !} ) exp [ - \frac{{\{ x - ( {E}_{0} - {\sum }_{i = 1}^{n} {v}_{i} {ω}_{i} ) \}}^{2}}{2 {C}^{2}} ] ,$$

where, I(x) denotes an intensity of absorption and emission of a wavelength x, E₀ denotes electron transition energy in a ground state and an excited state, Sᵢ denotes a Huang-Rhys factor that is a difference in molecular structure expressed by a specific vibrational mode, ωᵢ denotes a vibrational energy level of a specific vibrational mode, vᵢ denotes an energy level of a specific vibrational mode, C denotes a baseline distribution function parameter, and n denotes an approximated number of vibrational energy levels.

The parameters may include transition energy of the Franck-Condon progression, a baseline distribution function parameter, transition degrees and vibrational energy levels of each vibrational mode according to an approximated number of vibrational energy levels.

The molecular structure representation may be in a Molfile format or in a format of the simplified molecular-input line-entry system (SMILES).

The generated spectrum may include an emission or absorption spectrum according to a wavelength with respect to the organic molecule.

Other features and aspects will be apparent from the following detailed description, the drawings, and the claims.

### BRIEF DESCRIPTION OF THE DRAWINGS

FIG. 1 shows an example of applying the Franck-Condon principle to a neural network model, according to one or more embodiments.

FIG. 2 shows an example of predicting a spectrum of an organic molecule, according to one or more embodiments.

FIG. 3 shows an example method for training a neural network model for predicting a spectrum of an organic molecule, according to one or more embodiments.

FIG. 4 shows an example method for predicting a spectrum of an organic molecule, according to one or more embodiments.

FIG. 5 shows an example apparatus for predicting a spectrum of an organic molecule, according to one or more embodiments.

FIG. 6 shows an example of performance of a prediction apparatus, according to one or more embodiments.

Throughout the drawings and the detailed description, unless otherwise described or provided, the same or like drawing reference numerals will be understood to refer to the same or like elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known after an understanding of the disclosure of this application may be omitted for increased clarity and conciseness.

The features described herein may be embodied in different forms and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

The terminology used herein is for describing various examples only and is not to be used to limit the disclosure. The articles “a,” “an,” and “the” are intended to include the plural forms as well, unless the context clearly indicates otherwise. As used herein, the term “and/or” includes any one and any combination of any two or more of the associated listed items. As non-limiting examples, terms “comprise” or “comprises,” “include” or “includes,” and “have” or “has” specify the presence of stated features, numbers, operations, members, elements, and/or combinations thereof, but do not preclude the presence or addition of one or more other features, numbers, operations, members, elements, and/or combinations thereof.

Throughout the specification, when a component or element is described as being “connected to,” “coupled to,” or “joined to” another component or element, it may be directly “connected to,” “coupled to,” or “joined to” the other component or element, or there may reasonably be one or more other components or elements intervening therebetween. When a component or element is described as being “directly connected to,” “directly coupled to,” or “directly joined to” another component or element, there can be no other elements intervening therebetween. Likewise, expressions, for example, “between” and “immediately between” and “adjacent to” and “immediately adjacent to” may also be construed as described in the foregoing.

Although terms such as “first,” “second,” and “third”, or A, B, (a), (b), and the like may be used herein to describe various members, components, regions, layers, or sections, these members, components, regions, layers, or sections are not to be limited by these terms. Each of these terminologies is not used to define an essence, order, or sequence of corresponding members, components, regions, layers, or sections, for example, but used merely to distinguish the corresponding members, components, regions, layers, or sections from other members, components, regions, layers, or sections. Thus, a first member, component, region, layer, or section referred to in the examples described herein may also be referred to as a second member, component, region, layer, or section without departing from the teachings of the examples.

Unless otherwise defined, all terms, including technical and scientific terms, used herein have the same meaning as commonly understood by one of ordinary skill in the art to which this disclosure pertains and based on an understanding of the disclosure of the present application. Terms, such as those defined in commonly used dictionaries, are to be interpreted as having a meaning that is consistent with their meaning in the context of the relevant art and the disclosure of the present application and are not to be interpreted in an idealized or overly formal sense unless expressly so defined herein. The use of the term “may” herein with respect to an example or embodiment, e.g., as to what an example or embodiment may include or implement, means that at least one example or embodiment exists where such a feature is included or implemented, while all examples are not limited thereto.

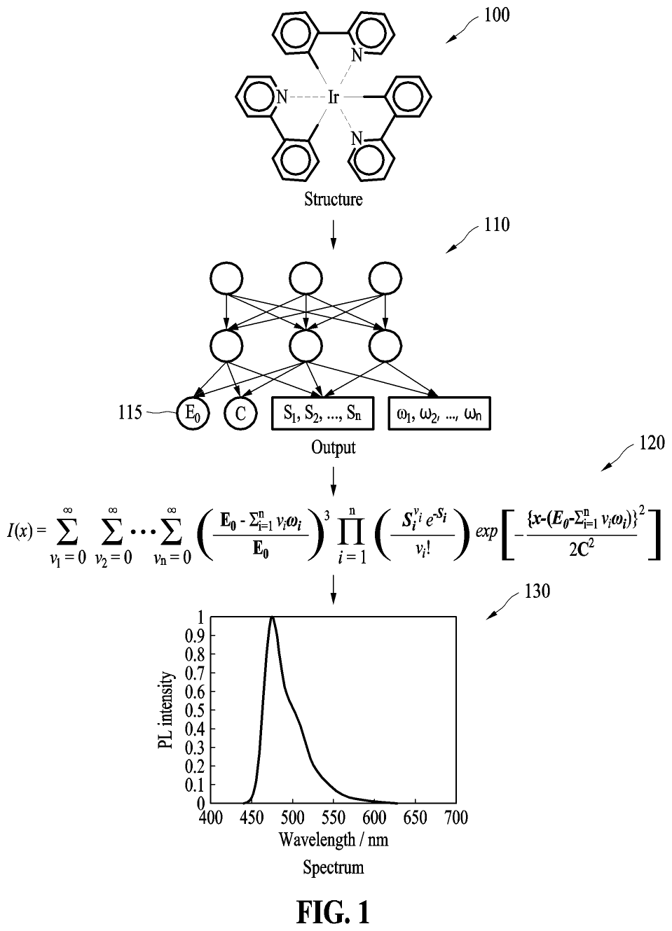

FIG. 1 shows an example of applying the Franck-Condon principle to a neural network model, according to one or more embodiments.

As an overview, a molecule structure representation 100 (describing a particular organic molecule) may be inputted to a spectrum prediction model 110 (e.g., a neural network model). The spectrum prediction model 110 may, as previously trained, perform inference on the molecule structure representation 100 and output an inference result 115 in the form of Franck-Condon approximation parameters. The predicted Franck-Condon approximation parameters may be applied to an algorithm 120 that implements the Franck-Condon equation. The algorithm 120, according to the predicted parameters, may output a wavelength spectrum 130 corresponding to the organic molecule represented by the molecule structure representation 100. Some explanation of the Franck-Condon principle follows.

When an organic molecule receives electrical energy or light energy, the organic molecule may transition from a ground state to an excited state. The electron transition from the ground state to the excited state can be understood by the Franck-Condon principle. According to the Franck-Condon principle, since an atomic nucleus has a significantly greater mass compared to an electron, the electron transition occurs rapidly and before the nucleus responds. Accordingly, an electric charge density in a molecule may vary according to the electron transition, and nuclei that have been stopped may vibrate by repeatedly advancing and receding as the nuclei are rapidly exposed to a new field of force.

A molecule in an excited state may include various vibrational modes, and each vibrational mode may include a unique vibrational energy level. In other words, when an electron transition from the ground state to the excited state occurs, the transition may occur by various vibrational modes and a vibrational energy level.

A value of a vibrational energy level may vary depending on a vibrational mode, and when an electron transition occurs, light of various wavelengths may be absorbed due to various vibrational energy level values, and as a result, an absorption spectrum may expand. Similarly, when electron transition from the excited state to the ground state occurs, the Franck-Condon principle may be applied and an emission spectrum may be widened since various vibrational modes and vibrational energy levels in the ground state exist. The light spectrum of absorption and emission may occur in a UV-Vis spectrum and an infrared (IR) spectrum.

The absorption and emission spectra according to a Franck-Condon analysis may be computed with the following Franck-Condon progression.

$$I ( x ) ∝ \cdot {\sum }_{{v}_{i}} {\prod }_{i = 1}^{n} ( \frac{{S}_{i}^{{v}_{i}} {e}^{- {S}_{i}}}{{v}_{i} !} ) Γ ( x - {E}_{0} \pm {\sum }_{i = 1}^{n} {v}_{i} {ω}_{i} ) . \qquad Equation 1$$

Equation 1 is a Franck-Condon progression. In Equation 1, I(x) denotes an absorption or emission intensity as a function of a specific wavelength x, E₀ denotes electron transition energy in a ground state or an excited state, Sᵢ denotes a Huang-Rhys factor (a difference in molecular structure expressed by a specific vibrational mode), and ωᵢ denotes a vibrational energy level in a specific vibrational mode. Γ denotes a line-shape function (a baseline distribution); generally, a Gaussian function may be used. The “±” indicates that addition or subtraction may be performed, depending on whether an emission spectrum or an absorption spectrum is being computed.

Equation 2 shows the Franck-Condon progression using a Gaussian function as a line-shape function. The algorithm 120 may implement Equation 2. Incidentally, although various equations such as Equation are mentioned herein, such equations are mathematical descriptive of the configuration of actual computing hardware/instructions. Computing hardware/instructions (code) may be realized using software/hardware engineering tools to create source code (or a high level circuit design), which the tools may readily translate to a specific circuit design, actual code (in the form of instructions executable by a processor), or the like. In other words, the mathematical description herein is a convenient language for describing the characteristics and actions of a computing system in a way that engineers can understand and use to construct a device or actual instructions that will operate as described herein.

$$I ( x ) = {\sum }_{{v}_{1} = 0}^{∞} {\sum }_{{v}_{2} = 0}^{∞} … {\sum }_{{v}_{n} = 0}^{∞} {( \frac{{E}_{0} - {\sum }_{i = 1}^{n} {v}_{i} {ω}_{i}}{{E}_{0}} )}^{3} {\prod }_{i = 1}^{n} ( \frac{{S}_{i}^{{v}_{i}} {e}^{- {S}_{i}}}{{v}_{i} !} ) exp [ - \frac{{\{ x - ( {E}_{0} - {\sum }_{i = 1}^{n} {v}_{i} {ω}_{i} ) \}}^{2}}{2 {C}^{2}} ] . \qquad Equation 2$$

C is a line-shape function parameter and the size of the line-shape function may vary depending on the value of C.

According to Equation 2, a continuous emission or absorption spectrum of an organic molecule may follow the Franck-Condon principle, and the continuous spectrum of the organic molecule in a wide area/range may be predicted by predicting some parameters including E₀, C, S₁, S₂, . . . , Sₙ, ω₁, ω₂, . . . , ωₙ in Equation 2. For example, the parameters may be predicted/output by the spectrum prediction model 110 (see “Output” in FIG. 1). To reiterate, the above parameters may be predicted as shown in FIG. 1. A spectrum may be predicted for a continuous wavelength by plugging the obtained parameters into Equation 2. The spectrum prediction model 110 may be a neural network model and may use a graph neural network, for example, a Dimenet network.

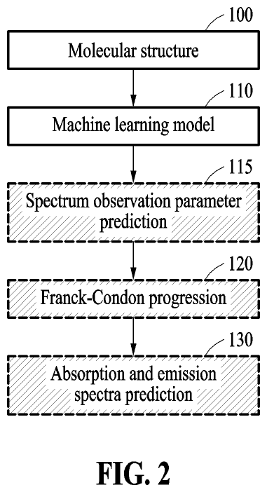

FIG. 2 shows an example of predicting a spectrum of an organic molecule, according to one or more embodiments.

When a molecular structure representation 100 of an organic molecule is input to a neural network model (e.g., the spectrum prediction model 110) that has been trained to derive a parameter (e.g., inference result 115) included in the Franck-Condon progression described above, a continuous spectrum of the organic molecule may be obtained.

Referring to Equation 2, the Franck-Condon progression (algorithm 120) may be approximated by adjusting Huang-Rhys factors S₁, S₂, . . . , Sₙ and the number of vibrational energy levels ω₁, ω₂, . . . , ωₙ among the parameters. For example, the Franck-Condon progression may be approximated to n=1, n=2, n=3, n=4, . . . etc., and as n increases, a more accurate prediction of the spectrum may be obtained. The following non-limiting examples are cases in which n=3.

Although the theoretical number of Huang-Rhys factors may be 3N-6 (N: the number of atoms in a molecule), in the actual calculation and prediction, the number thereof may be reduced and approximated for the efficiency of the neural network.

By setting n=3, the neural network model for predicting parameters for computing the spectrum of the organic molecule may be trained to derive eight parameters, which are E₀, C, S₁, S₂, S₃, ω₁, ω₂, and ω₃. The trained neural network model may infer eight parameter values with respect to the spectrum. An absorption spectrum or an emission spectrum depending on each wavelength may be obtained by applying the parameters to the approximated Franck-Condon progression. The + and − signs of the absorption spectrum and the emission spectrum may be reversed in Equation 1, as the case may be.

The obtained spectrum of the organic molecule may be in the form of a data structure (e.g., an array) containing intensities for discrete wavelength values (i.e., a spectrum distribution).

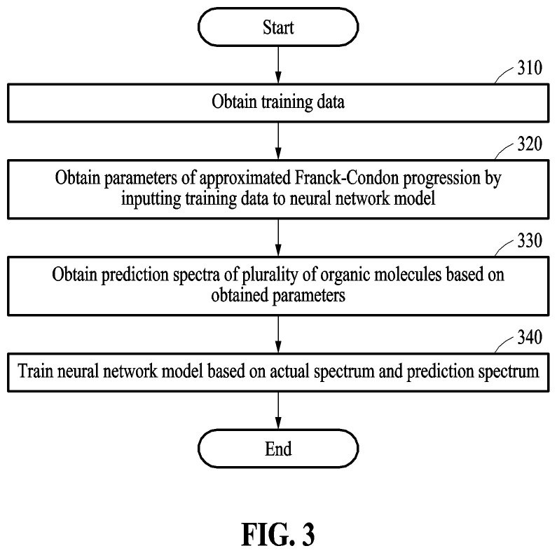

FIG. 3 shows an example method of training a neural network model (e.g., spectrum prediction model 110) for predicting a spectrum of an organic molecule, according to one or more embodiments.

A training device for training a neural network model for predicting a spectrum of an organic molecule (hereinafter, referred to as a “training device”) may train a neural network model through operations 310 to 330.

In operation 310, the training device may obtain training data, including molecular structures of organic molecules and respective actual spectra of the molecular structures of the organic molecules. The training data may be in the form of training samples, where each training sample is an actual spectrum (ground truth) paired with a corresponding molecular structure.

The organic molecule may be any molecule including carbon, and the shape thereof is not limited. The molecular structure of the organic molecule may be represented in the form of Molfile or simplified molecular-input line-entry system (SMILES), for example.

The actual spectrum of each training data sample may be a spectrum of values (intensities) in each wavelengths/wavebands and may be, for example, data obtained through experimentation (e.g., actual measurements).

Referring to a given training sample as representative of training for all of the training data, in operation 320, the training device may obtain parameters of the approximated Franck-Condon progression by inputting the given training data sample to the neural network model.

The training device may perform pre-training on the organic molecule to increase the accuracy of the parameters. For example, a highest occupied molecular orbital (homo) energy level, a lowest unoccupied molecular orbital (lumo) energy level, and an electron transition energy level may be calculated using a density functional theory (DFT) with respect to the organic molecules. In addition, fine-tuning may be performed.

The approximated Franck-Condon progression may correspond to Equation 2, and as an example, when n=3, eight parameters of E₀, C, S₁, S₂, S₃, ω₁, ω₂, and ω₃ may be inferred by the neural network model based on the molecule structure of the given training sample. Each parameter may denote transition energy of the Franck-Condon progression, a line-shape function parameter, a vibrational energy level of each vibrational mode according to the approximated number of vibrational energy levels (n=1, 2, 3), and a transition degree.

In operation 330, the training device may obtain a predicted spectrum of the organic molecule of the given training sample based on the obtained parameters, as described next with reference to the given training sample. The term “predicted spectrum” refers to the spectrum implicitly predicted via the obtained parameters; the spectrum may not be the immediate output of the neural network model.

The training device may apply the given training sample's obtained (inferred) parameters to the approximated Franck-Condon progression may obtain an absorption or emission spectrum for the entire area with respect to the organic molecules of the given training sample through the approximated Franck-Condon progression.

In operation 340, the training device may train the neural network model based on an error between the predicted spectrum and the actual spectrum.

Learning or updating of the neural network model may be performed based on a loss function using an error between the prediction spectrum calculated by the derived parameters and the actual (ground truth) spectrum (e.g., obtained through an actual observation). Equation 3 may express the loss function.

$$loss = \sqrt{\sum {( {y}_{prediction} - {y}_{actual} )}^{2}} = \sqrt{\sum {( {I ( x )}_{prediction} - {I ( x )}_{actual} )}^{2}} \qquad Equation 3$$

The training device may aggregate the errors between the actual spectrum and the prediction spectrum within a range of x=400 to 800 nm (a non-limiting example of the visible light spectrum) and use the aggregated errors as a loss function.

The training device may train the neural network model to derive parameters to minimize the loss function.

When a molecular structure of a new organic molecule is input, the trained neural network model may derive parameters with respect to a spectrum of the new organic molecule and may predict absorption and emission spectra of a continuous wavelength domain based on the parameters.

In another example, a function such as Lorenztian may be used rather than Gaussian as a line-shape function of the Franck-Condon progression. When the Lorenztian function is used as the line-shape function, the Franck-Condon progression may be approximated as Equation 4.

$$I ( x ) = {\sum }_{{v}_{1} = 0}^{∞} {\sum }_{{v}_{2} = 0}^{∞} … {\sum }_{{ν}_{n} = 0}^{∞} \frac{{( \frac{{E}_{0} - {\sum }_{i = 1}^{n} {v}_{i} {ω}_{i}}{{E}_{0}} )}^{3} {\prod }_{i = 1}^{n} ( \frac{{S}_{i}^{{v}_{i}} {e}^{- {S}_{i}}}{{v}_{i} !} )}{[ {\{ x - ( {E}_{0} - {\sum }_{i = 1}^{n} {v}_{i} {ω}_{i} ) \}}^{2} + {C}^{2} ]} \qquad Equation 4$$

Even if Equation 4 is used as the approximated Franck-Condon progression, the neural network model may be trained to derive parameters of E₀, C, S₁, S₂, . . . , Sₙ, ω₁, ω₂, . . . , ωₙ by using the same loss function.

In another example, a vibrational quantum number vᵢ may be limited to express absorption and emission spectra that are significant to the Franck-Condon progression. As the vibrational quantum number increases, the intensity of the absorption or emission spectrum may rapidly decrease. For example, Equation 5 shown below may correspond to the approximated Franck-Condon progression in which the vibrational quantum number is set to be calculated until 5.

$$I ( x ) = {\sum }_{{v}_{1} = 0}^{5} {\sum }_{{v}_{2} = 0}^{5} {\sum }_{{v}_{3} = 0}^{5} {( \frac{{E}_{0} - {\sum }_{i = 1}^{3} {v}_{i} {ω}_{i}}{{E}_{0}} )}^{3} {\prod }_{i = 1}^{3} ( \frac{{S}_{i}^{{v}_{i}} {e}^{- {S}_{i}}}{{v}_{i} !} ) exp [ - \frac{{\{ x - ( {E}_{0} - {\sum }_{i = 1}^{3} {v}_{i} {ω}_{i} ) \}}^{2}}{2 {C}^{2}} ] \qquad Equation 5$$

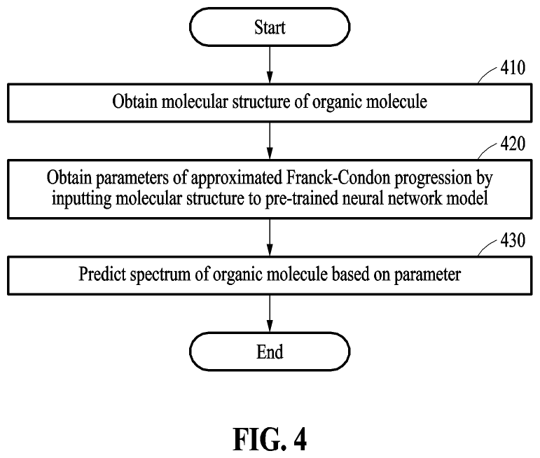

FIG. 4 shows an example method for predicting a spectrum of an organic molecule, according to one or more embodiments.

In operation 410, the prediction apparatus may obtain a molecular structure of an organic molecule.

The prediction apparatus may receive a molecular structure of a new organic molecule in the form of Molfile or SMILES; “new” meaning one not included in the training data.

In operation 420, the prediction apparatus may obtain/infer parameters of an approximated Franck-Condon progression by inputting the molecular structure to a pre-trained neural network model.

The pre-trained neural network model may correspond to a neural network model described with reference to FIGS. 1 to 3 (e.g., spectrum prediction model 110), and may have been trained based on an error between an actual (ground truth) spectrum of a molecular structure and a prediction spectrum of the approximated Franck-Condon progression (as performed for multiple such training samples).

The neural network model may be trained to predict parameters of the Franck-Condon progression. As a non-limiting example, the prediction apparatus may derive eight parameters, transition energy E₀ of the Franck-Condon progression, a line-shape function parameter C, and vibrational energy levels and transition degrees S₁, S₂, S₃, ω₁, ω₂, ω₃ depending on the approximated number of vibrational energy levels (n=1, 2, 3).

In operation 430, the prediction apparatus may generate a predicted spectrum of an organic molecule based on the obtained parameters.

The prediction apparatus may apply the parameters obtained through the neural network model to the approximated Franck-Condon progression and may thereby obtain an absorption or emission spectrum of the entire wavelength area with respect to the new organic molecule. The neural network model may be a graph neural network and a predicted spectrum may be expressed as a graph. For example, the neural network model may have an layers of nodes, including an input layer, one or more hidden layers, and an output layer. Nodes in each layer may have weighted connections to an adjacent layer. The input layer may have nodes configured to receive molecular models. The output layer may have nodes configured to output the aforementioned parameters. The training of the neural network model may include backpropagating error through the output layer using, for example, the gradient descent technique.

The neural network model for predicting a spectrum of an organic molecule may be used to develop a machine learning algorithm for developing an organic photodiode (OPD), organic photovoltaics (OPV), and an organic light-emitting diode (OLED) material, for example.

The neural network model may be produced as a separate machine learning algorithm software and may be used to predict properties related to the absorption and emission of a molecule by predicting the entire absorption and emission spectra of an organic molecule.

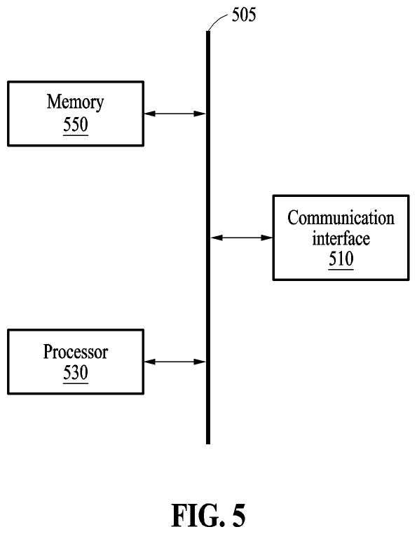

FIG. 5 shows an example of a prediction apparatus for predicting a spectrum of an organic molecule, according to one or more embodiments.

Referring to FIG. 5, a prediction apparatus 500 in one embodiment may include a communication interface 510, a processor 530, and a memory 550. The communication interface 510, the processor 530, and the memory 550 may communicate with each other via a communication bus 505.

The communication interface 510 may receive a molecular structure of an organic molecule to predict a spectrum.

The processor 530 may predict a spectrum with respect to the molecular structure of the organic molecule received through the communication interface 510. The spectrum may be calculated by the approximated Franck-Condon progression expressed based on a pre-trained parameter through a training device. The processor 530 may be any of, or any combination of, the processors described below, or otherwise.

The memory 550 may store a variety of information generated in the processing process of the processor 530 described above. In addition, the memory 550 may store a variety of data and programs. The memory 550 may include volatile memory or non-volatile memory. The memory 550 may include a large-capacity storage medium such as a hard disk to store a variety of data.

In addition, the processor 530 may perform at least one method described with reference to FIGS. 1 through 4 or an algorithm corresponding to the at least one method. The processor 530 may be a hardware-implemented data processing device with a circuit that is physically structured to execute the desired operations. For example, the desired operations may include code or instructions in a program. The processor 530 may be implemented as, for example, a central processing unit (CPU), a graphics processing unit (GPU), or a neural network processing unit (NPU). For example, the prediction apparatus 500 that is implemented as hardware may include, for example, a microprocessor, a central processing unit (CPU), a processor core, a multi-core processor, a multiprocessor, an ASIC, and/or an FPGA.

The processor 530 may execute the program and control the prediction apparatus 500. Program codes to be executed by the processor 530 may be stored in the memory 550.

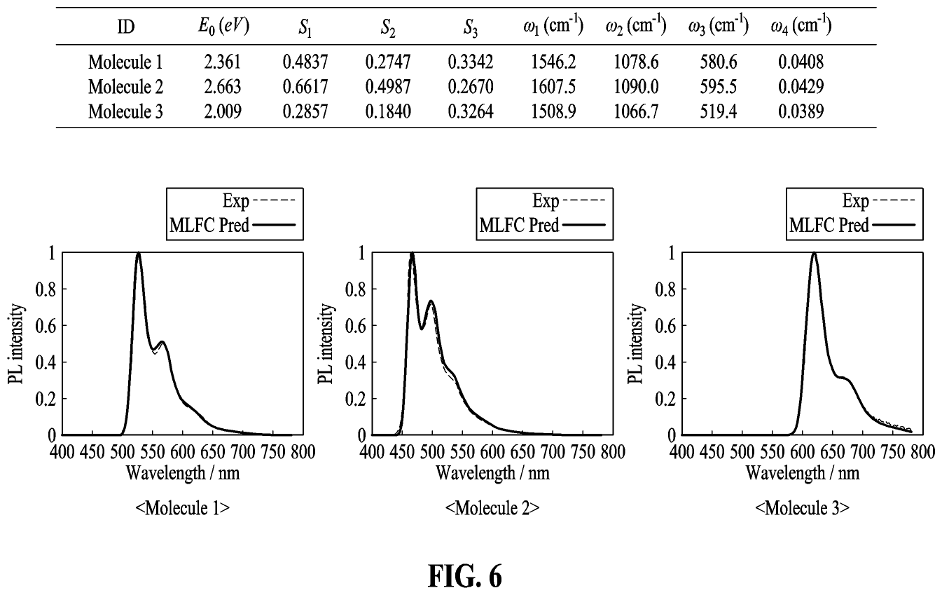

FIG. 6 shows an example of performance of a prediction apparatus, according to one or more embodiments.

The graph illustrates an example of an emission spectrum predicted with respect to a specific organic molecule. Values of the predicted spectrum with respect to wavelengths in a 400 to 800 nm wavelength domain are illustrated as a graph.

A Franck-Condon progression may be approximated to derive vibrational modes and vibrational energy level parameters S₁ to S₃, ω₁ to ω₃. As shown in the drawings, a continuous spectrum may be predicted and the similarity may be high in comparison to the actual spectrum. A spectrum value may be expressed by an intensity value between 0 and 1.

The computing apparatuses, the processors, the memories, the displays, the information output system and hardware, the storage devices, and other apparatuses, devices, units, modules, and components described herein with respect to FIGS. 1-6 are implemented by or representative of hardware components. Examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. A hardware component may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 1-6 that perform the operations described in this application are performed by computing hardware, for example, by one or more processors or computers, implemented as described above implementing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions herein, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media. Examples of a non-transitory computer-readable storage medium include read-only memory (ROM), random-access programmable read only memory (PROM), electrically erasable programmable read-only memory (EEPROM), random-access memory (RAM), dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above disclosure, the scope of the disclosure may also be defined by the claims and their equivalents, and all variations within the scope of the claims and their equivalents are to be construed as being included in the disclosure.

What is claimed is:

1. A method performed by a computing device comprising processing hardware and storage hardware, the method comprising:
storing, in the storage hardware, a representation of a molecular structure of an organic molecule;
performing, by a neural network model executed by the processing hardware, inference on the representation of the molecular structure, to infer parameters of an approximated Franck-Condon progression; and
generating a spectrum of the organic molecule by the processing hardware applying the inferred parameters to the approximated Franck-Condon progression.

2. The method of claim 1, further comprising training the neural network model based on an error between a ground truth spectrum of the representation of the molecular structure and the generated spectrum of the organic molecule.

3. The method of claim 1, wherein, with respect to organic molecules, the neural network model is trained after calculating a highest occupied molecular orbital (homo) energy level, a lowest unoccupied molecular orbital (lumo) energy level, and an electron transition energy level through a density functional theory (DFT).

4. The operating method of claim 1, wherein the processing hardware is configured to perform the following as the approximated Franck-Condon progression:
$$I ( x ) = {\sum }_{{v}_{1} = 0}^{∞} {\sum }_{{v}_{2} = 0}^{∞} … {\sum }_{{v}_{n} = 0}^{∞} {( \frac{{E}_{0} - {\sum }_{i = 1}^{n} {v}_{i} {ω}_{i}}{{E}_{0}} )}^{3} {\prod }_{i = 1}^{n} ( \frac{{S}_{i}^{{v}_{i}} {e}^{- {S}_{i}}}{{v}_{i} !} ) exp [ - \frac{{\{ x - ( {E}_{0} - {\sum }_{i = 1}^{n} {v}_{i} {ω}_{i} ) \}}^{2}}{2 {C}^{2}} ] ,$$
where, I(x) denotes an intensity of absorption and emission of a wavelength x, E₀ denotes electron transition energy in a ground state and an excited state, Sᵢ denotes a Huang-Rhys factor that is a difference in molecular structure expressed by a specific vibrational mode, ωᵢ denotes a vibrational energy level of a specific vibrational mode, vᵢ denotes an energy level of a specific vibrational mode, C denotes a baseline distribution function parameter, and n denotes an approximated number of vibrational energy levels.

5. The operating method of claim 1, wherein the parameters comprise transition energy of the Franck-Condon progression, a baseline distribution function parameter, transition degrees, and vibrational energy levels of each vibrational mode according to the approximated number of vibrational energy levels.

6. The operating method of claim 1, wherein the molecular structure is in a Molfile format or a format of the simplified molecular-input line-entry system (SMILES).

7. The operating method of claim 1, wherein the spectrum of the organic molecule comprises an emission or absorption spectrum according to a wavelength with respect to the organic molecule.

8. A method of training a neural network model performed by a computing device comprising processing hardware and storage hardware, the method comprising:
accessing, by the processing hardware, training data in the storage hardware, the training data comprising training samples, each training sample comprising a molecular structure representation of an organic molecule and an associated ground truth spectrum of the molecular structure;
for each training sample, generating, by the processing hardware, corresponding parameters of an approximated Franck-Condon progression by inputting the molecular structure representation of the training sample to a neural network model which infers the corresponding parameters;
for each training sample, generating, by the processing hardware, a corresponding spectrum of the training sample based on the corresponding obtained parameters; and
for each training sample, training, by the processing hardware, the neural network model based on an error between the corresponding ground truth spectrum and the corresponding generated spectrum.

9. The method of claim 8, wherein the processing hardware is configured to perform the following as the approximated Franck-Condon progression:
$$I ( x ) = {\sum }_{{v}_{1} = 0}^{∞} {\sum }_{{v}_{2} = 0}^{∞} … {\sum }_{{v}_{n} = 0}^{∞} {( \frac{{E}_{0} - {\sum }_{i = 1}^{n} {v}_{i} {ω}_{i}}{{E}_{0}} )}^{3} {\prod }_{i = 1}^{n} ( \frac{{S}_{i}^{{v}_{i}} {e}^{- {S}_{i}}}{{v}_{i} !} ) exp [ - \frac{{\{ x - ( {E}_{0} - {\sum }_{i = 1}^{n} {v}_{i} {ω}_{i} ) \}}^{2}}{2 {C}^{2}} ] ,$$
where, I(x) denotes an intensity of absorption and emission of a wavelength x, E₀ denotes electron transition energy in a ground state and an excited state, Sᵢ denotes a Huang-Rhys factor that is a difference in molecular structure expressed by a specific vibrational mode, ωᵢ denotes a vibrational energy level of a specific vibrational mode, vᵢ denotes an energy level of a specific vibrational mode, C denotes a baseline distribution function parameter, and n denotes an approximated number of vibrational energy levels.

10. The method of claim 8, wherein the parameters comprise transition energy of the Franck-Condon progression, a baseline distribution function parameter, transition degrees, and vibrational energy levels of each vibrational mode according to an approximated number of vibrational energy levels.

11. The method of claim 8, wherein the obtaining of the parameters for each of the training samples comprises:
calculating a highest occupied molecular orbital (homo) energy level, a lowest unoccupied molecular orbital (lumo) energy level, and an electron transition energy level using a density functional theory (DFT) for the organic molecules.

12. The method of claim 8, wherein the molecular structure representation of each training sample is in a Molfile format or a format of the simplified molecular-input line-entry system (SMILES).

13. The method of claim 1, wherein the neural network model comprises layers of nodes with weights of connections therebetween, the layers including an input layer configured to receive the molecule structure representations and an output layer configured to output the parameters.

14. An apparatus comprising:
one or more processors; and
a memory storing instructions configured to cause the one or more processors to:
access a molecular structure representation of an organic molecule;
generate parameters of an approximated Franck-Condon progression by inputting the molecular structure representation to a neural network model that infers the parameters from the molecular structure representation; and
generate a spectrum of the organic molecule based on the generated parameters.

15. The apparatus of claim 14, wherein the neural network model is trained based on an error between a ground truth spectrum of the molecular structure representation and the generated spectrum.

16. The apparatus of claim 14, wherein, with respect to organic molecules, the neural network model is trained after calculating a highest occupied molecular orbital (homo) energy level, a lowest unoccupied molecular orbital (lumo) energy level, and an electron transition energy level through a density functional theory (DFT).

17. The apparatus of claim 14, wherein the instructions are further configured to cause the one or more processors to perform the following approximated Franck-Condon progression:
$$I ( x ) = {\sum }_{{v}_{1} = 0}^{∞} {\sum }_{{v}_{2} = 0}^{∞} … {\sum }_{{v}_{n} = 0}^{∞} {( \frac{{E}_{0} - {\sum }_{i = 1}^{n} {v}_{i} {ω}_{i}}{{E}_{0}} )}^{3} {\prod }_{i = 1}^{n} ( \frac{{S}_{i}^{{v}_{i}} {e}^{- {S}_{i}}}{{v}_{i} !} ) exp [ - \frac{{\{ x - ( {E}_{0} - {\sum }_{i = 1}^{n} {v}_{i} {ω}_{i} ) \}}^{2}}{2 {C}^{2}} ] ,$$
where, I(x) denotes an intensity of absorption and emission of a wavelength x, E₀ denotes electron transition energy in a ground state and an excited state, Sᵢ denotes a Huang-Rhys factor that is a difference in molecular structure expressed by a specific vibrational mode, ωᵢ denotes a vibrational energy level of a specific vibrational mode, vᵢ denotes an energy level of a specific vibrational mode, C denotes a baseline distribution function parameter, and n denotes an approximated number of vibrational energy levels.

18. The apparatus of claim 14, wherein the parameters comprise transition energy of the Franck-Condon progression, a baseline distribution function parameter, transition degrees and vibrational energy levels of each vibrational mode according to an approximated number of vibrational energy levels.

19. The apparatus of claim 14, wherein the molecular structure comprises representation may be in a Molfile format or in a format of the simplified molecular-input line-entry system (SMILES).

20. The apparatus of claim 14, wherein the generated spectrum comprises an emission or absorption spectrum according to a wavelength with respect to the organic molecule.
